(19) United States
(12) Patent Application Publication
(10) Pub. No.: US 2008/0166821 A1
(43) Pub. Date: Jul. 10, 2008

(54) Immunochromatography kit

(75) Inventors: Takayoshi Oyamada, Kanagawa (JP); Junichi Katada, Saitama-ken (JP)

Correspondence Address: BIRCH STEWART KOLASCH & BIRCH, PO BOX 747, FALLS CHURCH, VA, 22040-0747, US

(73) Assignee: FUJIFILM Corporation, Tokyo (JP)

(21) Appl. No.: 11/979,847

(22) Filed: Nov. 8, 2007

(30) Foreign Application Priority Data
Nov. 8, 2006 (JP) 2006-302842

(51) Int. Cl. G01N 33/536

(52) U.S. Cl. 436/536

(57) ABSTRACT

An immunochromatography kit including organic silver salt particles, a reducing agent for silver ions, and a metal colloid label or a metal sulfide label. The immunochromatography kit uses an immunoreaction of an analyte and an antibody or antigen that can bind specifically thereto, and analyzes a signal from a label derived from an immune complex.

### CROSS-REFERENCE TO RELATED APPLICATION

This application claims priority under 35USC 119 from Japanese Patent Application No. 2006-302842, the disclosure of which is incorporated by reference herein.

### BACKGROUND OF THE INVENTION

1. Field of the Invention

The present invention relates to an immunochemical analytical material with which an analyte-containing sample can be qualitatively and quantitatively analyzed easily, promptly and accurately.

2. Description of the Related Art

Among biologically active substances or environmental pollutants such as natural products, toxins, hormones and agricultural chemicals, there are numerous substances acting in an ultratrace amount. Accordingly, instrumental analytical methods capable of high-sensitivity analysis have conventionally been widely used for qualitative and quantitative measurement of these substances. However, instrumental analytical methods are poor in specificity, require time for analysis including pretreatment of a sample, and are troublesome in operation. Thus instrumental analytical methods are inconvenient for the purpose of rapid and easy measurements for which there have been needs in recent years. On the other hand, immunological measuring methods are highly specific and much easier in operation than instrumental analytical methods. Therefore immunological measuring methods have gradually spread in the field of measurement of biologically active substances and environmental pollutants. However, conventional immunological measuring methods such as enzyme immunoassays and latex agglutination assays using 96-well plates do not always provide satisfactory rapidness and easiness of measurement or detection sensitivity.

There are also needs for improvement of the sensitivity of tests which currently use relatively invasive samples such swab and blood, the result of which is expected to realize less burdensome tests to patients in which a very small amount of an analyte contained in less invasive samples such as snot, mouth wash and urine is detected.

In recent years, examination kits using immunochromatography (referred to hereinafter as immunochromatography kit) have been used more often in examination of infections which requires particularly rapid diagnosis. According to spread of these kits, patients with infections can be identified using a rapid and easy method, and subsequent diagnosis and therapy can be conducted immediately and accurately. For example, in immunochromatography utilizing the sandwich method, a labeled second antibody capable of binding to an analyte (for example, an antigen), and a sample solution which may possibly contain the analyte, are developed in an insoluble thin film-shaped support (for example, a glass fiber membrane, a nylon membrane or a cellulose membrane) on which a first antibody capable of specifically binding to the analyte was immobilized in a specific region. As a result, an immune complex with the analyte is formed on the region of the insoluble thin film-shaped support which region has the first antibody immobilized thereon. The analyte can be measured by detecting a signal such as color development or coloring of a label. The label may be, for example, a protein such as an enzyme, colored latex particles, metal colloids, or carbon particles.

Immunochromatography does not require any massive facilities or instruments for judgment and measurement. Further, immunochromatography is simple in operation and promptly gives measurement results by dropping a sample solution which may possibly contain an analyte and leaving it for about 5 to 10 minutes. For this reason, this technique is used widely as easy, rapid and highly specific methods of judgment and measurement in many scenes, for example, for clinical examination in hospitals and in assays in laboratories.

Among biologically active substances or environmental pollutants such as natural products, toxins, hormones and agricultural chemicals, there are many substances that are effective even in ultratrace amounts that are undetectable by conventional common immunochromatography. Therefore, there are demands for development of rapid, easy and highly sensitive immunochromatography methods.

A large number of techniques attempting at higher sensitivity have conventionally been disclosed, such as techniques of a innovated means of development (see, for example, Japanese Patent Application (JP-A) No. 1-32169 and JP-A No. 4-299262), techniques of innovated colored particles (see, for example, JP-A No. 5-10950 and JP-A No. 5-133956), techniques of innovated member for development (see, for example, JP-A No. 7-318560), techniques utilizing an avidin-biotin bond (see, for example, JP-A No. 10-68730), techniques utilizing an enzyme immunoassay (see, for example, JP-A No. 11-69996), techniques using catalytically active metal colloids (see, for example, JP-A No. 2003-262638), and techniques of precipitating metal ions (see, for example, JP-A No. 2002-202307).

However there are needs for still higher sensitivity although the immunochromatography approaches the enzyme immunoassays due to increased sensitivity in the detection of an analyte achieved by these techniques.

### SUMMARY OF THE INVENTION

The present invention has been made in view of the above circumstances and provides an immunochromatography kit.

An aspect of the present invention provides an immunochromatography kit including organic silver salt particles, a reducing agent for silver ions, and a metal colloid label or metal sulfide label. The immunochromatography kit uses an immune reaction of an analyte and an antibody or antigen that binds specifically to the analyte and analyzes a signal from a label derived from the immobilized immune complex.

### BRIEF DESCRIPTION OF THE DRAWINGS

Exemplary embodiments of the present invention will be described in detail based on the following figures, wherein:

FIG. 1 is a plane view schematically illustrating an embodiment of a comparative immunochromatography kit;

FIG. 2 is a schematic longitudinal sectional view schematically illustrating the immunochromatography kit illustrated in FIG. 1; and

FIG. 3 is a schematic longitudinal sectional view schematically illustrating the immunochromatography kit according to the present invention.

### DETAILED DESCRIPTION OF THE INVENTION

In general, the detection sensitivity of conventional immunochromatography in the case of bacteria is 10⁵ to 10⁷ CFU/ml. The gene amplification method (PCR method) is mentioned as a recent highly sensitive detection method, which has achieved a detection sensitivity of up to 10³ to 10⁴ CFU/ml. However, the PCR method needs massive facilities and instruments and complicated operation. Moreover, the PCR method requires a long time (several hours) until detection, and thus cannot be considered to be an easy and rapid measurement method. If the sensitivity of conventional immunochromatography is heightened by about 1- to 4-digits, examinations which have conventionally been carried out by the PCR method and which has not been easy and rapid, are expected to be conducted easily and rapidly.

There is also demand for higher sensitivity in examination of infections for which immunochromatographic measurement methods have been established. For example, the examination of influenza by immunochromatography has spread widely in recent years as an easy and rapid examination method; however, re-examination is necessary in some cases because the result could be false negative due to poor detection sensitivity when the amount of the virus in an initial stage of infection is relatively low. In general, influenza virus is considered to grow 10-fold in 4 hours. Therefore improvement of the sensitivity by 1-digit, for example, enables the infection to be judged four hours earlier than conventional methods. From the viewpoint of reducing the burden of patients attending a hospital many times, there is demand for an easy and rapid examination method, such as immunochromatography, with higher sensitivity.

1. Immunochromatography

In general, immunochromatography is a technique in which an analyte is measured and determined easily, rapidly and specifically by the following method. That is, a chromatographic carrier having at least one reaction site containing an immobilizing reagent (such as antibody or antigen) capable of binding to the analyte is used as a solid phase. While a dispersion liquid that includes a detection label dispersed therein modified with a reagent capable of binding to the analyte moves chromatographically on the chromatographic carrier as a mobile phase, the analyte binds specifically to the detection label and reaches the reaction site. The analyte-detection label complex binds specifically to the immobilizing reagent at the reaction site. Therefore the detection label is concentrated at the immobilization reagent part only when the analyte is present in a test solution. The presence of the analyte in the test solution is determined qualitatively and quantitatively through visual inspection of the concentrated detection label or detection with a suitable instrument.

The immunochromatography kit according to the present invention includes an organic silver salt and a reducing agent for silver ions. The signal is amplified by an amplification reaction using the analyte-detection label complex that is bonded to the immobilizing reagent as a core, resulting in achievement of higher sensitivity. According to the invention, it is possible to provide a simpler and quicker high-sensitivity immunochromatography kit that does not require supply, from the outside, of metal ions or a reducing agent solution for amplification, which is required in conventional immunochromatography kits.

2. Specimen

The specimen to be analyzed with the immunochromatography kit according to the present invention is not particularly limited insofar as it is a sample that possibly contains an analyte of interest. The sample may be a biological sample, examples of which include animal (human in particular) body fluids (for example, blood, serum, blood plasma, spinal fluid, lacrimal fluid, sweat, urine, pus, snot or sputum), excrements (for example, feces), organs, tissues, mucosae and skin, swabs that may possibly contain such samples, mouth washes, animals or plants themselves and dried materials thereof.

3. Pretreatment of the Specimen

The specimen to be examined with the immunochromatography kit according to the present invention may be an intact specimen, or in the form of an extract obtained by extracting the specimen with a suitable extraction solvent, or in the form of a diluted solution obtained by diluting the extract with a suitable diluent, or in the form of a concentrate obtained by concentrating the extract by a suitable method. The extraction solvent to be used may be, for example, a solvent used in an ordinary immunological analysis method (for example, water, physiological saline or a buffer solution) or a water-miscible organic solvent in which the antigen-antibody reaction can be directly carried out after dilution with a solvent that may be selected from those described above.

4. Configuration

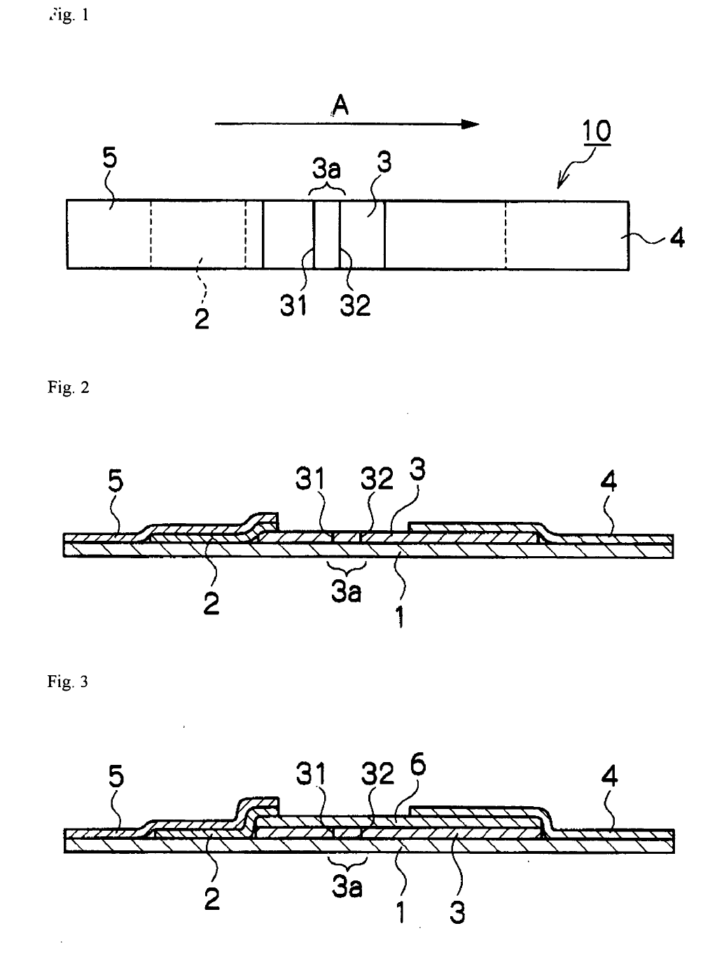

Immunochromatographic strips usable in the immunochromatography kit according to the present invention is not particularly limited insofar as they are immunochromatographic strips usable in ordinary immunochromatography. By way of example, FIG. 1 is a schematic plane view of a conventional immunochromatographic strip. FIG. 2 is a schematic longitudinal sectional view of the immunochromatography kit shown in FIG. 1. FIG. 3 is a schematic longitudinal sectional view of the immunochromatographic strip according to the invention.

In the immunochromatographic strip 10 according to the invention, a sample addition pad 5, a labeled substance-holding pad (for example, a gold colloid antibody-holding pad) 2, a chromatographic carrier (for example, an antibody-immobilized membrane) 3, and an absorbent pad 4 are arranged, from the upstream to downstream in the direction of development (direction indicated by arrow A in FIG. 1), on a pressure-sensitive adhesive sheet 1.

The chromatographic carrier 3 has a capture site 3a and has a detection zone (also referred to as a detection part) 31 that is a region on which an antibody or antigen capable of specifically binding to the analyte is immobilized. If desired, the chromatographic carrier 3 may further have a control zone (also referred to as a control part) 32 that is a region on which a control antibody or antigen is immobilized. The detection zone 31 and control zone 32 contain an organic silver salt for amplification and a reducing agent for silver ions.

The labeled substance-holding pad 2 may be prepared by preparing a suspension containing a label, then applying the suspension onto a suitable absorbent pad (for example, a glass fiber pad) followed by drying.

For example, a glass fiber pad can be used as the sample addition pad 5.

4-1. Detection Label

As the detection label, colored particles used in immune agglutination may be used. For example, metals such as metal colloids may be used. The average particle diameter of carrier particles (or colloids) is preferably in the range of 0.02 to 10 μm. Dye-containing liposomes or microcapsules are also usable as the colored particles. Any conventionally known colored metal colloids may be used as colored particles for labeling. Examples thereof include gold colloids, silver colloids, platinum colloids, iron colloids, aluminum hydroxide colloids, and composite colloids thereof. Preferable examples include gold colloids, silver colloids, platinum colloids, and composite colloids thereof. In particular, gold colloids and silver colloids are preferable in that the gold colloids at a suitable particle diameter show red color and silver colloids at a suitable particle diameter show yellow color. The average particle diameter of a metal colloid is preferably from about 1 nm to about 500 nm, more preferably from 5 nm to 100 nm at which a particularly strong color tone may be obtained. Binding of the metal colloid to the specifically binding substance may be conducted by a method known in the art (for example, The Journal of Histochemistry and Cytochemistry, Vol. 30, No. 7, pp. 691-696 (1982)). That is, the metal colloid and the specifically binding substance (for example an antibody) are mixed in a suitable buffer solution at room temperature for 5 minutes or more. After the reaction, the precipitate obtained by centrifugation is dispersed in a solution containing a dispersant such as polyethylene glycol, whereby the desired metal colloid-labeled specific binding substance can be obtained. When gold colloid particles are used as the metal colloid, commercially available gold colloid particles may be used. As an alternative, gold colloid particles may be prepared by a common method, for example a method of reducing chlorauric acid with sodium citrate (Nature Phys. Sci., vol. 241, 20 (1973) etc.).

According to the invention, in the immunochromatography kit using, as a detection label, a metal colloid label, metal sulfide label or another metal alloy label (also referred to hereinafter as a metallic label) or a metal-containing polymer particle label, the signal from the metallic label can be amplified. Specifically, after formation of a complex of the analyte and the detection label, silver ions supplied from the organic silver salt and a reducing agent for silver ions are contacted therewith; as a result, the silver ions are reduced by the reducing agent to form silver particles, which deposit on the metallic label as a core, whereby the metallic label is amplified to enable high-sensitivity analysis of the analyte. Accordingly conventionally known immunochromatography can be applied, as it is, to the immunochromatography kit according to the invention except that the precipitation reaction of silver particles generated by reduction of silver ions with the reducing agent is carried out on the label of the immune complex so as to analyze thus amplified signal.

In the immunochromatography kit according to the present invention, a metal colloid label or a metal sulfide label may be used as the label for labeling an antibody or antigen which can bind specifically to an analyte (antigen or antibody) or for labeling a standard compound. The metal colloid label or metal sulfide label is not particularly limited insofar as it is a label usable in the ordinary immunochromatography. Examples of the metal colloid label include platinum colloids, gold colloids, palladium colloids and silver colloids, and mixtures thereof. Examples of the metal sulfide label include sulfides of iron, silver, palladium, lead, copper, cadmium, bismuth, antimony, tin and mercury. In the immunochromatography kit according to the invention, one of these metal colloid labels and/or metal sulfide labels may be used as the label, or two or more of these metal colloid labels and/or metal sulfide labels may be used as the label.

4-2. Antibody

In the immunochromatography kit according to the present invention, the antibody having specificity for an analyte is not particularly limited; for example, it is possible to use an antiserum prepared from serum of an animal immunized with the analyte, an immunoglobulin fraction purified from the antiserum, a monoclonal antibody obtained by cell fusion using splenocytes of the animal immunized with the analyte, or fragments thereof (for example, F(ab′)2, Fab, Fab′ or Fv). Preparation of such an antibody may be carried out by a common method.

4-3. Chromatographic Carrier

The chromatographic carrier is preferably a porous carrier, particularly preferably a nitrocellulose membrane, a cellulose membrane, an acetyl cellulose membrane, a polysulfone membrane, a polyether sulfone membrane, a nylon membrane, glass fibers, a nonwoven fabric, a cloth, threads or the like.

Usually, a substance for detection is immobilized in a part of the chromatographic carrier to form a detection zone. The substance for detection may be directly immobilized through physical or chemical bonds onto a part of the chromatographic carrier; as an alternative, the substance for detection may be bound physically or chemically onto fine particles such as latex particles and then immobilized by trapping the fine particles onto a part of the chromatographic carrier. Prior to use, the chromatographic carrier after immobilizing the substance for detection thereon is preferably subjected to a treatment for preventing unspecific adsorption. The treatment may be conducted by using an inert protein, etc.

4-4. Sample Addition Pad

Examples of the materials for the sample addition pad include, but are not limited to, those having uniform characteristics, such as a cellulose filter paper, glass fibers, polyurethane, polyacetate, cellulose acetate, nylon and a cotton cloth. The sample addition part not only functions to receive the analyte-containing sample that is added, but also functions to filter off insoluble particles etc. in the sample. The material constituting the sample addition part may be used after being subjected to treatment for preventing unspecific adsorption in order to prevent deterioration in analysis accuracy due to unspecific adsorption of the analyte in the sample onto the material of the sample addition part.

4-5. Labeled Substance Holding Pad

Examples of the material of the labeled substance holding pad include, for example, a cellulose filter paper, glass fibers and a nonwoven fabric. The labeled substance holding pad is prepared by impregnating the pad with a predetermined amount of the detection label prepared as described above, followed by drying.

4-6. Absorbent Pad

The absorbent pad constitutes a portion where the added sample is physically absorbed due to chromatographic migration and where an unreacted label etc. that is not immobilized on the detection part of the chromatographic carrier is removed by absorption. The material for the absorbent pad may be a water-absorbing material such as a cellulose filter paper, a nonwoven fabric, a cloth or cellulose acetate. Because the chromatographic speed of the chromatographic leading end of the added sample after reaching the absorbing portion varies depending on the material and size of the absorbent material, an adequate speed for the measurement of the analyte can be set by selection of the absorbent material.

5. Immunoassay Method

Hereinafter, in reference to specific embodiments where the immunochromatography used in the invention is applied to the sandwich method, antibody immobilizing competitive method, antigen immobilizing competitive method and immobilized antigen method, the immunochromatography used in the invention is described in that order.

5-1. Sandwich Method

In an embodiment where the sandwich method is applied to the immunochromatography kit according to the present invention (hereinafter referred to simply as the sandwich method), the analysis of the analyte may be conducted, for example, in the following procedures though the procedures are not limited thereto. Initially, first and second antibodies having specificity for an analyte (antigen) are prepared in advance by the method described above. In addition, the second antibody is labeled in advance. The first antibody is immobilized on a suitable insoluble thin film-shaped support (for example, a nitrocellulose membrane, a glass fiber membrane, a nylon membrane or a cellulose membrane) and is brought into contact with a test sample (or its extract) that may possibly contain the analyte (antigen), so that an antigen-antibody reaction occurs if the analyte is present in the test sample. This antigen-antibody reaction may be carried out in the same manner as in usual antigen-antibody reaction. During or after the antigen-antibody reaction, the sample is brought into contact further with an excess amount of the labeled second antibody, so that an immune complex of the immobilized first antibody—the analyte (antigen)-labeled second antibody forms when the analyte is present in the sample.

In the sandwich method, after the completion of the reaction of the immobilized first antibody, the analyte (antigen) and the second antibody, the labeled second antibody not involving in the formation of the immune complex is removed. Subsequently, for example, the signal from the label of the labeled second antibody involved in the immune complex may be amplified by supplying metal ions and a reducing agent to that region of the insoluble thin film-shaped support on which the immobilized first antibody is immobilized. As an alternative, the signal from the label of the labeled second antibody involved in the immune complex may be amplified by adding metal ions and a reducing agent to the labeled second antibody, and adding the labeled second antibody, the metal ions and the reducing agent simultaneously to the thin film-shaped support.

5-2. Antibody Immobilizing Competitive Method

In an embodiment where the antibody immobilizing competitive method is applied to the immunochromatography kit according to the present invention (hereinafter referred to simply as the antibody immobilizing competitive method), the analysis of the analyte may be conducted, for example, in the following procedures though the procedures are not limited thereto. First, an antibody having specificity for an analyte (antigen) is prepared in advance by the method described above, and the antibody is immobilized on a suitable insoluble thin film-shaped support (for example, a nitrocellulose membrane, a glass fiber membrane, a nylon membrane or a cellulose membrane). Separately, the standard compound is labeled in advance. The labeled standard compound and a test sample (or its extract) that may possibly contain the analyte (antigen) are developed and brought into contact with each other, during or after which the labeled standard compound is developed and brought into contact with the immobilized antibody, so that an antigen-antibody reaction occurs if the analyte is present in the sample. This antigen-antibody reaction may be carried out in the same manner as in usual antigen-antibody reaction.

In the antibody immobilizing competitive method, after the reaction of the immobilized antibody on the insoluble thin film-shaped support and the labeled standard compound (that is, the labeled antigen) is completed, the labeled standard compound bonded to the immobilized antibody and the labeled standard compound not bonded to the immobilized antibody are separated from each other. Subsequently, for example, metal ions and a reducing agent may be added to the region of the insoluble thin film-shaped support on which the immobilized antibody is immobilized, so that the signal from the label of the labeled antigen bonded to the immobilized antibody is amplified. As an alternative, metal ions and a reducing agent may be added to the labeled standard compound, and the labeled standard compound, the metal ions and the reducing agent may be added simultaneously to the thin film-shaped support, so that the signal from the label of the labeled standard compound bonded to the immobilized antibody is amplified. The above separation may be carried out for example by washing with a buffer solution.

5-3. Antigen Immobilizing Competitive Method

In an embodiment where the antigen immobilizing competitive method is applied to the immunochromatography kit according to the present invention (hereinafter referred to simply as the antigen immobilizing competitive method), the analysis of the analyte may be conducted, for example, in the following procedures though the procedures are not limited thereto. First, an antibody having specificity for an analyte (antigen) is prepared in advance by the method described above. The antibody is labeled in advance. Further, a known amount of the standard compound (antigen) is immobilized on a suitable insoluble thin film-shaped support (for example, a nitrocellulose membrane, a glass fiber membrane, a nylon membrane or a cellulose membrane).

In the antigen immobilizing competitive method, after completion of the reaction of the immobilized standard compound (that is, the immobilized antigen) on the insoluble thin film-shaped support and the labeled antibody, the labeled antibody bonded to the immobilized standard compound and the labeled antibody not bonded to the immobilized standard compound are separated from each other. Subsequently, for example, the signal from the label of the labeled antibody bonded to the immobilized standard compound may be amplified by supplying metal ions and a reducing agent to the region of the insoluble thin film-shaped support on which the immobilized standard compound is immobilized. As an alternative, metal ions and a reducing agent may be added to the labeled antibody, and the labeled antibody, the metal ions and the reducing agent may be simultaneously added to the thin film-shaped support, so that the signal from the label of the labeled standard antibody bonded to the immobilized standard compound is amplified. The above-mentioned separation may be attained by, for example, washing with a buffer solution.

5-4. Immobilized Antigen Method

In an embodiment where the immobilized antigen method is applied to the immunochromatography kit according to the present invention (hereinafter referred to simply as the immobilized antigen method), the analysis of the analyte may be conducted, for example, in the following procedures though the procedures are not limited thereto. First, a second antibody having specificity for an analyte (antibody) is prepared in advance by the method described above. The second antibody is labeled in advance. The antigen to which the analyte (antibody) binds specifically is immobilized on a suitable insoluble thin film-shaped support (for example, a nitrocellulose membrane, a glass fiber membrane, a nylon membrane or a cellulose membrane) and then brought into contact with a test sample (or its extract) that may possibly contain the analyte (antibody), so that an antigen-antibody reaction occurs if the analyte is present in the sample. This antigen-antibody reaction may be carried out in the same manner as in usual antigen-antibody reaction. During or after the antigen-antibody reaction, the sample is brought into contact further with an excess amount of the labeled second antibody, so that an immune complex of the immobilized antigen-analyte (antibody)-labeled second antibody forms if the analyte is present in the sample.

In the immobilized antigen method, after the completion of the reaction of the immobilized antigen, the analyte (antibody) and the second antibody, the labeled second antibody not involved in the immune complex is removed. Then, for example, metal ions and a reducing agent may be supplied to a region of the insoluble thin film-shaped support on which the immobilized antigen is immobilized, so that the signal from the label of the labeled second antibody involved in the immune complex is amplified. As an alternative, metal ions and a reducing agent may be added to the labeled second antibody, and the labeled second antibody, the metal ions and the reducing agent may be simultaneously added to the thin film-shaped support, so that the signal from the label of the labeled second antibody involved in the immune complex is amplified.

6. Organic Silver Salt

The organic silver salt used in the present invention is an organic compound containing a reducible silver ion. Preferably, the organic silver salt is a silver salt or coordination compound which forms metallic silver upon heating to 50° C. or more in the presence of a reducing agent wherein the metallic silver is relatively stable against light. The organic silver salt used in the invention is preferably a compound selected from a silver salt of an azole compound or a silver salt of a mercapto compound. The azole compound is preferably a nitrogen-containing heterocyclic compound, more preferably a triazole compound or a tetrazole compound. The mercapto compound is a compound having at least one mercapto or thion group in its molecule.

The silver salt of the nitrogen-containing heterocyclic compound in the present invention is preferably a silver salt of a compound having an imino group. Typical compounds include, but are not limited to, a silver salt of 1,2,4-triazole and silver salts of benzotriazole and derivatives thereof (for example, a methylbenzotriazole silver salt or 5-chlorobenzotriazole silver salt), 1H-tetrazole compounds such as phenyl mercaptotetrazole described in U.S. Pat. No. 4,220,709 and imidazole and imidazole derivatives described in U.S. Pat. No. 4,260,677. Among these silver salts, particularly preferable compounds are silver salts of benzotriazole derivatives or mixtures of two or more thereof.

The silver salt of the nitrogen-containing heterocyclic compound used in the invention is most preferably a silver salt of a benzotriazole derivative.

The compound having a mercapto or thion group in the present invention is preferably a heterocyclic compound containing 5 or 6 atoms. At least one of the atoms in the cycle in this compound is a nitrogen atom, and the other atoms in the cycle are selected from carbon, oxygen and sulfur atoms. Example of the heterocyclic compound include, but are not limited to, triazoles, oxazoles, thiazoles, thiazolines, imidazoles, diazoles, pyridines, and triazines.

Typical examples of the silver salt of the compound having a mercapto or thion group include, but are not limited to, 3-mercapto-4-phenyl-1,2,4-triazole silver salt, 2-mercapto-benzimidazole silver salt, 2-mercapto-5-aminothiazole silver salt, 2-(2-ethylglycolamide)benzothiazole silver salt, 5-carboxy-1-methyl-2-phenyl-4-thiopyridine silver salt, mercaptotriazine silver salt, 2-mercaptobenzoxazole silver salt, silver salts of compounds (for example, 1,2,4-mercaptothiazole derivative silver salt, 3-amino-5-benzylthio-1,2,4-thiazole silver salt) described in U.S. Pat. No. 4,123,274, and thion compound silver salts (for example, 3-(2-carboxyethyl)-4-methyl-4-thiazoline-2-thion silver salt described in U.S. Pat. No. 3,785,830).

A compound not containing a heterocycle is also usable as the compound having a mercapto or thion group in the invention. The mercapto or thion derivative not containing a heterocycle is preferably an aliphatic or aromatic hydrocarbon compound having 10 or more carbon atoms in total.

Among mercapto or thion derivatives not containing a heterocycle, examples of useful compounds include, but are not limited to, silver thioglycolate (for example, silver S-alkylthioglycolate having a C12 to C22 alkyl group) and silver dithiocarboxylate (for example, silver dithioacetate or thioamide silver salt).

An organic compound having a silver carboxylate is also preferable, which may be, for example, a linear carboxylate. Specifically, a C6 to C22 carboxylate is preferable. In addition, a silver aromatic carboxylate is also preferable. Examples of aromatic carboxylates and other carboxylates include, but are not limited to, substituted or unsubstituted silver benzoates (for example, silver 3,5-dihydroxybenzoate, silver o-methylbenzoate, silver m-methylbenzoate, silver p-methylbenzoate, silver 2,4-dichlorobenzoate, silver acetamidobenzoate and silver p-phenylbenzoate), silver tannate, silver phthalate, silver terephthalate, silver salicylate, silver phenylacetate and silver pyromellitate.

In the present invention, thioether group-containing fatty acid silver as described in U.S. Pat. No. 3,330,663 may also be used preferably. Soluble silver carboxylates having an ether or thioether linkage-containing hydrocarbon chain or having a sterically shielded substituent at the α-position (on a hydrocarbon group) or at the ortho-position (on an aromatic group) are also usable. These compounds have improved solubility in a coating solvent so that they are able to form a coating with less light scattering.

Such silver carboxylates are described, for example, in U.S. Pat. No. 5,491,059. Any mixtures of silver salts described therein may be used as necessary in the present invention.

Silver sulfonates described in U.S. Pat. No. 4,504,575 are also usable in embodiments of the present invention. Silver sulfosuccinates described in EP-A 0227141 are also useful in the invention.

Acetylene silver salts described in, for example, U.S. Pat. Nos. 4,761,361 and 4,775,613 are also usable in the present invention. The silver salts may also be provided as core/shell-type silver salts described in U.S. Pat. No. 6,355,408. These silver salts are composed of a core containing one or more silver salts and a shell containing one or more different silver salts.

Another useful non-photosensitive silver source in the present invention is a silver dimer synthetic product composed of two different silver salts described in U.S. Pat. No. 6,472,131. Such nonphotosensitive silver dimer synthetic product is composed of two different silver salts. When the two silver salts contain linear saturated hydrocarbon groups as silver ligands, the difference in the number of carbon atoms between the ligands is 6 or more.

The organic silver salt is contained generally in an amount of 0.001 mol/m² to 0.2 mol/m², preferably 0.001 mol/m² to 0.05 mol/m², in terms of the silver amount.

7. Reducing Agent

As the reducing agent for silver ion, any material capable of reducing silver(I) ion into silver may be used.

Developing agents (for example, methyl gallate, hydroquinone, substituted hydroquinone, 3-pyrazolidones, p-aminophenols, p-phenylenediamines, hindered phenols, amidoximes, azines, catechols, pyrogallols, ascorbic acid (or derivatives thereof) and leuco dyes) used in wet-process silver halide photosensitive materials, or other materials evident for those skilled in the art (see, for example, U.S. Pat. No. 6,020,117 (Bauer et al.)) may be used in the present invention.

The term “ascorbic acid reducing agent” refers to ascorbic acid or a derivative thereof. Ascorbic acid reducing agents are described in many literatures as described below, including, for example, U.S. Pat. No. 5,236,816 (Purol et al.) and literatures cited therein.

The reducing agent in the present invention is preferably an ascorbic acid reducing agent. Useful ascorbic acid reducing agents include ascorbic acid, analogues thereof, isomers thereof and derivatives thereof. Examples of such compounds include, but are not limited to, D- or L-ascorbic acid and sugar derivatives thereof (for example, sorboascorbic acid, gamma-lactoascorbic acid, 6-desoxy-L-ascorbic acid, L-rhamnoascorbic acid, imino-6-desoxy-L-ascorbic acid, glucoascorbic acid, fucoascorbic acid, glucoheptoascorbic acid, maltoascorbic acid, L-arabosascorbic acid), sodium ascorbate, potassium ascorbate, isoascorbic acid (or L-erythroascorbic acid) and salts thereof (for example, alkali metal salts, ammonium salts or salts known in the art), endiol-containing ascorbic acid, enaminol-containing ascorbic acid, and thioenol-containing ascorbic acid, for example compounds described in U.S. Pat. No. 5,498,511, EP-A 0585,792, EP-A 0573700, EP-A 0588408, U.S. Pat. Nos. 5,089,819, 5,278,035, 5,384,232, 5,376,510, JP 7-56286, U.S. Pat. No. 2,688,549 and Research Disclosure 37152 (March, 1995).

Among these compounds, D-, L-, or D,L-ascorbic acid (and alkali metal salts thereof) and isoascorbic acid (and alkali metal salts thereof) are preferable, and sodium salts are preferable. If necessary, a mixture of two or more such reducing agents may be used.

A hindered phenol may be preferably used singly or in combination with one or more gradation-hardening reducing agents and/or contrast enhancers. A hindered phenol is a compound having only one hydroxyl group on a benzene ring and at least one substituent at the ortho-position relative to the hydroxyl group. The hindered phenol reducing agent may have plural hydroxyl groups insofar as the hydroxyl groups are located on different benzene rings.

Examples of the hindered phenol reducing agent include binaphthols (that is, dihydroxybinaphthols), biphenols (that is, dihydroxybiphenols), bis(hydroxynaphthyl)methanes, bis(hydroxyphenyl)methanes (i.e., bisphenols), hindered phenols and hindered naphthols, each of which may be substituted.

Typical binaphthols include, but are not limited to, 1,1′-bi-2-naphthol, 1,1′-bi-4-methyl-2-naphthol, 6,6′-dibromo-bi-2-naphthol, and compounds described in U.S. Pat. Nos. 3,094,417 and 5,262,295.

Typical biphenols include, but are not limited to, 2,2′-dihydroxy-3,3′-di-t-butyl-5,5′-dimethylbiphenyl, 2,2′-dihydroxy-3,3′,5,5′-tetra-t-butylbiphenyl, 2,2′-dihydroxy-3,3′-di-t-butyl-5,5′-dichlorobiphenyl, 2-(2-hydroxy-3-t-butyl-5-methylphenyl)-4-methyl-6-n-hexylphenol, 4,4′-dihydroxy-3,3′,5,5′-tetra-t-butylbiphenyl, 4,4′-dihydroxy-3,3′,5,5′-tetramethylbiphenyl, and compounds described in U.S. Pat. No. 5,262,295.

Typical bis(hydroxynaphthyl)methanes include, but are not limited to, 4,4′-methylenebis(2-methyl-1-naphthol) and compounds described in U.S. Pat. No. 5,262,295.

Typical bis(hydroxyphenyl)methanes include, but are not limited to, bis(2-hydroxy-3-t-butyl-5-methylphenyl)methane (CAO-5), 1,1′-bis(2-hydroxy-3,5-dimethylphenyl)-3,5,5-trimethyl hexane (NONOX or PERMANAX WSO), 1,1′-bis(3,5-di-t-butyl-4-hydroxyphenyl)methane, 2,2′-bis(4-hydroxy-3-methylphenyl)propane, 4,4′-ethylidene-bis(2-t-butyl-6-methylphenol), 2,2′-isobutylidene-bis(4,6-dimethylphenol) (LOWINOX 221B46), 2,2′-bis(3,5-dimethyl-4-hydroxyphenyl)propane, and compounds described in U.S. Pat. No. 5,262,295.

Typical hindered phenols include, but are not limited to, 2,6-di-t-butylphenol, 2,6-di-t-butyl-4-methylphenol, 2,4-di-t-butylphenol, 2,6-dichlorophenol, 2,6-dimethylphenol, and 2-t-butyl-6-methylphenol.

Typical hindered naphthols include, but are not limited to, 1-naphthol, 4-methyl-1-naphthol, 4-methoxy-1-naphthol, 4-chloro-1-naphthol, 2-methyl-1-naphthol, and compounds described in U.S. Pat. No. 5,262,295.

Other compounds disclosed as reducing agents include amidoximes (for example, phenylamidoxime), 2-thienylamidoxime, p-phenoxyphenylamidoxime, azines (for example, 4-hydroxy-3,5-dimethoxybenzaldehydrazine), a combination of an aliphatic carboxylic allyl hydrazide and ascorbic acid (for example, a combination of 2,2′-bis(hydroxymethyl)-propionyl-β-phenyl hydrazide and ascorbic acid), a combination of a polyhydroxybenzene and at least one of hydroxylamine, reductone or hydrazine (for example, a combination of hydroquinone and bis(ethoxyethyl)hydroxylamine), piperidi-4-methylphenylhydrazine, hydroxamic acids (for example, phenylhydroxamic acid, p-hydroxyphenylhydroxamic acid, and o-alaninehydroxamic acid), a combination of an azine and a sulfonamidophenol (for example, a combination of phenothiazine and 2,6-dichloro-4-benzenesulfonamidophenol), α-cyanophenylacetic acid derivatives (for example, ethyl-α-cyano-2-methylphenylacetic acid, ethyl-α-cyanophenylacetic acid), bis-o-naphthol (for example, 2,2′-dihydroxy-1-binaphthyl, 6,6′-dibromo-2,2′-dihydroxy-1,1′-binaphthyl, bis(2-hydroxy-1-naphthyl)methane), a combination of bis-o-naphthol and a 1,3-dihydroxybenzene derivative (for example, 2,4-dihydroxybenzophenone, 2,4-dihydroxyacetophenone), 5-pyrazolones (for example, 3-methyl-1-phenyl-5-pyrazolone), reductones (for example, dimethylaminohexose reductone, anhydrodihydro-aminohexose reductone, or anhydrodihydro-piperidone-hexose reductone), sulfonamidophenol reducing agents (for example, 2,6-dichloro-4-benzenesulfonamidophenol, p-benzenesulfonamidophenol), indane-1,3-diones (for example, 2-phenylindane-1,3-dione), chromans (for example, 2,2-dimethyl-7-t-butyl-6-hydroxychroman), 1,4-dihydroxypyridines (for example, 2,6-dimethoxy-3,5-dicarbetoxy-1,4-dihydropyridine), ascorbic acid derivatives (1-ascorbic palmitate, ascorbic stearate), unsaturated aldehydes (ketones), and 3-pyrazolidones.

As the reducing agent usable in the present invention, substituted hydrazines may be mentioned such as sulfonyl hydrazines described in U.S. Pat. No. 5,464,738. Other useful reducing agents are described in, for example, U.S. Pat. Nos. 3,074,809, 3,094,417, 3,080,254 and 3,887,417. Auxiliary reducing agents descried in U.S. Pat. No. 5,981,151 are also useful.

The reducing agent may be a combination of a hindered phenol reducing agent and one or more compounds selected from various auxiliary reducing agents such as those mentioned below. A mixture of this combination plus a contrast enhancer (that is, a mixture of the 3 components) is also useful. As the auxiliary reducing agent, it is possible to use trityl hydrazide and formyl-phenyl hydrazide described in U.S. Pat. No. 5,496,695.

A contrast enhancer may be used in combination with the reducing agent. Useful contrast enhancers include, but are not limited to, hydroxylamines (including hydroxylamine, alkyl-substituted derivatives thereof and aryl-substituted derivatives thereof), alkanolamines and phthalic ammonium described in U.S. Pat. No. 5,545,505, hydroxamic acid compounds described in U.S. Pat. No. 5,545,507, N-acylhydrazine compounds described in U.S. Pat. No. 5,558,983, and hydrogen atom donor compounds described in U.S. Pat. No. 5,637,449.

Not all combinations of reducing agents and organic silver salts are equally effective. One preferable combination is a combination of benzotriazole silver salt, a substituted compound thereof or a mixture thereof as the organic silver salt and an ascorbic acid-based reducing agent as the reducing agent.

The reducing agent in the present invention may be contained in an amount of 1 mass % to 10 mass % (dry mass) based on the amount of silver in the organic silver. When the reducing agent is added to a layer other than the organic silver-containing layer in a multilayer structure, the amount of the reducing agent is slightly higher and is desirably from about 2 mass % to about 15 mass %. An auxiliary reducing agent is contained in an amount of about 0.001 mass % to 1.5 mass % (dry weight).

The kit according to the present invention is preferably heated after development of the sample to be examined. The heating temperature is preferably in the range of 40° C. to 90° C., and the heating time is preferably in the range of 1 second to 120 seconds.

Exemplary embodiments of the invention are described below.

<1> An immunochromatography kit including organic silver salt particles, a reducing agent for silver ions, and a metal colloid label or a metal sulfide label, wherein the immunochromatography kit analyzes a signal from a label derived from an immune complex that is immobilized by using an immunoreaction of an analyte and an antibody or antigen capable of binding specifically to the analyte.

<2> The immunochromatography kit according to the above-mentioned <1>, which includes a solvent for the organic silver salt particles.

<3> The immunochromatography kit according to the above-mentioned <1>, wherein the organic silver salt particles include silver carboxylate or a nitrogen-containing heterocyclic silver salt.

<4> The immunochromatography kit according to the above-mentioned <3>, wherein the nitrogen-containing heterocyclic silver salt is a triazole compound or a tetrazole compound.

<5> The immunochromatography kit according to <3>, wherein the nitrogen-containing heterocyclic silver salt is a silver salt of a benzotriazole compound.

<6> The immunochromatography kit according to the above-mentioned <1>, wherein the phase transition temperature of the organic silver salt is from 40 to 100° C.

<7> The immunochromatography kit according to the above-mentioned <1>, which includes, in a single component part of the kit, the organic silver salt particles, the reducing agent for silver ions and the metal colloid label or the metal sulfide label.

<8> The immunochromatography kit according to the above-mentioned <7>, which includes, in the single component part of the kit, a first layer containing the organic silver salt particles and the reducing agent for silver ions and a second layer containing the metal colloid label or the metal sulfide label.

<9> The immunochromatography kit according to the above-mentioned <1>, wherein the metal colloid is a gold colloid, a silver colloid, a platinum colloid or a composite colloid thereof.

<10> The immunochromatography kit according to the above-mentioned <1>, wherein the average particle diameter of the metal colloid is from 5 to 100 nm.

<11> The immunochromatography kit according to the above-mentioned <1>, wherein the antibody or antigen is immobilized on a support.

<12> The immunochromatography kit according to the above-mentioned <11>, wherein the immune complex comprises the antibody or antigen, the analyte and an additional labeled antibody or antigen.

<13> The immunochromatography kit according to the above-mentioned <11>, wherein the immune complex comprises the antibody or antigen and the analyte, wherein the analyte is labeled.

### EXAMPLES

### Example 1

In Example 1, it is demonstrated that the immunochromatography kit according to the present invention is highly sensitive in a hCG detection system.

1. Preparation of a Gold Colloid (Detection Label) Modified with an Anti-hCG Antibody

1 ml of 50 mM KH₂PO₄ buffer (pH 7.0) was added to 9 ml of a gold colloid solution containing colloidal particles having a diameter of 50 nm (trade name: EM. GC50, manufactured by BBI Co., Ltd.), so that the pH was adjusted. 1 ml of 50 μg/ml anti-hCG antibody (trade name: ANTI-HCG 5008 SP-5 manufactured by Medix Biochemica) was added to the gold colloid solution, and then the mixture was stirred. After the mixture was left for 10 minutes, 550 μL of 1 mass % solution of polyethylene glycol (trade name: PEG, Mw. 20000, Product No. 168-11285, manufactured by Wako Pure Chemical Industries, Ltd.) was added thereto, and the resultant mixture was stirred. Thereafter, 1.1 ml of 10 mass % bovine serum albumin (trade name: BSA FRACTION V, Product No. A-7906, manufactured by SIGMA) aqueous solution was added thereto, and the resultant mixture was stirred. The solution was centrifuged at 8000 G at 4° C. for 30 minutes by a centrifuge (trade name: HIMAC CF16RX, manufactured by Hitachi, Ltd.), and the supernatant was removed so that about 1 ml remained. Then the gold colloid was dispersed again with an ultrasonic washing machine. Thereafter, the resultant dispersion was dispersed in 20 ml of a stock solution for gold colloid (20 mM Tris-HCl buffer, pH 8.2, 0.05 mass % PEG (Mw. 20000), 150 mM NaCl, 1 mass % BSA, 0.1 mass % NaN₃) and centrifuged again at 8000 G at 4° C. for 30 minutes. Then the supernatant was removed so that about 1 ml remained. The gold colloid was dispersed again with an ultrasonic washing machine to give an antibody-modified gold colloid (average diameter: 50 nm) solution.

2. Preparation of a Gold Colloid Antibody-Holding Pad

Each antibody-modified gold colloid prepared in item 1 above was diluted with a coating solution for gold colloid (20 mM Tris-HCl buffer, pH 8.2, 0.05 mass % PEG (Mw. 20000), 5 mass % sucrose) and water such that the OD at 520 nm became 1.5. This solution was uniformly applied to glass fiber pads (trade name: GLASS FIBER CONJUGATE PAD, manufactured by Millipore) cut in the size of 8 mm×150 mm in an amount of 0.8 ml per pad, and then dried overnight under reduced pressure to give a gold colloid antibody-holding pad.

3. Preparation of an Antibody-Immobilized Membrane (Chromatographic Carrier)

An antibody-immobilized membrane was prepared in the following manner by immobilizing an antibody on a nitrocellulose membrane (HIFLOW PLUS HF120 with a plastic lining, manufactured by Millipore) cut in the size of 25 mm×200 mm. Using a coater of inkjet type (BioDot Ltd.), the membrane with one of its long sides directed downwards was coated, at a position of 8 mm from the bottom, with an anti-hCG monoclonal antibody for immobilization (ANTI-ALPHA SUBUNIT 6601 SPR-5, manufactured by Medix Biochemica) prepared at a concentration of 0.5 mg/ml, thereby forming a coating in a line shape with a width of about 1 mm (“detection part”). In a similar manner, the membrane was coated, at a position of 12 mm from the bottom, with a control anti-mouse IgG antibody (anti-mouse IgG (H+L), rabbit F(ab′)2, Product No. 566-70621, Wako Pure Chemical Industries, Ltd.) prepared at a concentration of 0.5 mg/ml, thereby forming a line-shaped coating (“control part”). The coated membrane was dried at 50° C. for 30 minutes with a hot-air dryer. The membrane was immersed in 500 ml of a blocking solution (50 mM borate buffer, pH 8.5, containing 0.5 mass % casein (milk-derived product, Product No. 030-01505, manufactured by Wako Pure Chemical Industries, Ltd.)) in a vat, and was left therein for 30 minutes. Thereafter, the membrane was transferred to and immersed in 500 ml of a washing-stabilizing solution (0.5 mass % sucrose, 0.05 mass % sodium cholate, 50 mM Tris-HCl, pH 7.5) in a similar vat, and was left therein for 30 minutes. The membrane was taken out of the solution, and was dried overnight at room temperature to give an antibody-immobilized membrane.

4. Preparation of an Organic Silver Salt-Containing Sheet

### 4-1. Preparation of a Material for Coating

1) Preparation of a Fatty Acid Silver Salt Dispersion

### <Preparation of a Fatty Acid Silver Salt Dispersion>

150 g of lauric acid, 422 ml of distilled water, 49.2 ml of a 5 mol/L aqueous NaOH, and 120 ml of t-butyl alcohol were mixed and allowed to react at 75° C. for 1 hour under stirring to give a solution of sodium laurate. Separately, 206.2 ml of an aqueous solution containing 40.4 g of silver nitrate (pH 4.0) was prepared and kept at 1° C. A reaction container containing 635 ml of distilled water and 30 ml of t-butyl alcohol was kept at 30° C., and the whole of the previous sodium laurate solution and the whole of the aqueous silver nitrate solution were added thereto over 93 minutes and 15 seconds, and 90 minutes, respectively, at constant flow rates under sufficient stirring. During the addition, the aqueous silver nitrate solution only was added for first 11 minutes after the start of the addition of the aqueous silver nitrate solution, and thereafter, the addition of the sodium laurate solution was started, and after the addition of the silver nitrate aqueous solution was finished, the sodium laurate solution only was added for 14 minutes and 15 seconds. At this time, the temperature in the reaction container was kept at 30° C. and the liquid temperature was kept constant by controlling the external temperature. The piping for the addition of the sodium laurate solution was kept warm by circulating warm water between the pipes in a double pipe, such that the liquid temperature at the outlet of the addition nozzle was 75° C. The piping for the addition of the silver nitrate aqueous solution was kept warm by circulating cold water between the pipes in a double pipe. The addition position of the sodium laurate solution and the addition position of the silver nitrate aqueous solution were symmetrically arranged with respect to the stirring axis as the center, and were at such heights as to not contact with the reaction solution.

After the addition of the sodium laurate solution was finished, the mixture was stirred at the same temperature for 20 minutes, and the temperature was raised over 30 minutes to 35° C., followed by aging for 210 minutes. Just after the aging was finished, solids were separated by centrifugal filtration, and the solids were washed with water until the conductivity of filtrate water became 30 μS/cm. The fatty acid silver salt was obtained in this manner. The resultant solids were stored in the form of a wet cake without drying. When the form of the resultant silver laurate particles was evaluated by electron microscopy, the particles were crystals with a variation coefficient of sphere-equivalent diameter of 11%, an average aspect ratio of 1.9, and the following dimensions on average: thickness=0.1 μm, length=0.2 μm and width=0.2 μm.

19.3 kg polyvinyl alcohol (trade name: PVA-217) and water were added to the wet cake corresponding to 260 kg dry solid, so as to make the total weight 1000 kg. Then the mixture was converted into slurry with a dissolver blade and preliminarily dispersed with a pipeline mixer (trade name: PM-10, manufactured by Mizuho Industrial Co., Ltd.).

Then, the preliminarily dispersed stock solution was treated three times at a regulated pressure of 1150 kg/cm² with a dispersing machine (trade name: MICROFLUIDIZER M-610, manufactured by Microfluidex International Corporation, using a Z-type interaction chamber) to give a silver behenate dispersion. In cooling operation, the temperature of the dispersion was set at 18° C. by regulating the temperature of a cooling medium in corrugated-tube-type heat exchangers disposed before and after the interaction chamber.

2) Preparation of a Dispersion of a Reducing Agent

### <<Preparation of a Dispersion of a Reducing Agent-2>>

10 kg of water was added to, and mixed well with, 10 kg of reducing agent-2 (6,6′-di-t-butyl-4,4′-dimethyl-2,2′-butylidene diphenol) and 16 kg of 10 mass % aqueous solution of modified polyvinyl alcohol (trade name: POVAL MP203 manufactured by Kuraray Co., Ltd.), to prepare slurry. This slurry was sent via a diaphragm pump and dispersed for 3.5 hours with a horizontal sand mill (trade name: TVM-2, manufactured by AIMEX) charged with zirconia beads having an average diameter of 0.5 mm, and then 0.2 g of benzoisothiazolinone sodium salt and water were added thereto such that the concentration of the reducing agent became 25 mass %. This dispersion was heated at 40° C. for 1 hour and then heat-treated at 80° C. for 1 hour to give a dispersion of the reducing agent-2. The particles of the reducing agent contained in the reducing agent dispersion thus obtained had a median diameter of 0.50 μm and a maximum particle diameter of 1.6 μm or less. The resultant reducing agent dispersion was filtered through a polypropylene filter having a pore diameter of 3.0 μm to remove foreign substances such as contaminants, and then stored.

3) Preparation of a Dispersion of a Hydrogen Bonding Compound-1

10 kg of water was added to, and mixed well with, 10 kg of a hydrogen bonding compound-1 (tri(4-t-butylphenyl)phosphine oxide) and 16 kg of 10 mass % aqueous solution of modified polyvinyl alcohol (trade name: POVAL MP203 manufactured by Kuraray Co., Ltd.), to prepare slurry. This slurry was sent via a diaphragm pump and dispersed for 4 hours with a horizontal sand mill (trade name: UVM-2, manufactured by AIMEX) charged with zirconia beads having an average diameter of 0.5 mm, and then 0.2 g of benzoisothiazolinone sodium salt and water were added thereto such that the concentration of the hydrogen bonding compound became 25 mass %. This dispersion was heated at 40° C. for 1 hour and then heated at 80° C. for 1 hour to give a dispersion of the hydrogen bonding compound-1. The particles of the hydrogen bonding compound contained in the hydrogen bonding compound dispersion thus obtained had a median diameter of 0.45 μm and a maximum particle diameter of 1.3 μm or less. The resultant hydrogen bonding compound dispersion was filtered through a polypropylene filter having a pore diameter of 3.0 μm to remove foreign substances such as contaminants, and then stored.

4) Preparation of a Dispersion of a Reduction Accelerator-1

10 kg of water was added to, and mixed well with, 10 kg of a reduction accelerator-1 and 20 kg of 10 mass % aqueous solution of modified polyvinyl alcohol (trade name: POVAL MP203 manufactured by Kuraray Co., Ltd.), to prepare slurry. This slurry was sent via a diaphragm pump and dispersed for 3.5 hours with a horizontal sand mill (trade name: UVM-2, manufactured by AIMEX) charged with zirconia beads having an average diameter of 0.5 mm, and then 0.2 g of benzoisothiazolinone sodium salt and water were added thereto such that the concentration of the reduction accelerator became 20 mass %, to give a dispersion of the development accelerator-1. The particles of the reduction accelerator contained in the reduction accelerator dispersion thus obtained had a median diameter of 0.48 μm and a maximum particle diameter of 1.4 μm or less. The resultant reduction accelerator dispersion was filtered through a polypropylene filter having a pore diameter of 3.0 μm to remove foreign substances such as foreign particles, and then stored.

5) Preparation of a Dispersion of a Reduction Accelerator-2

A solid dispersion of a reduction accelerator-2 was also dispersed in a similar manner to the dispersing of the reduction accelerator-1, so that a 20 mass % dispersion was obtained.

6) Preparation of Polyhalogen Compounds

### <<Preparation of a Dispersion of an Organic Polyhalogen Compound-1>>

10 kg of an organic polyhalogen compound-1 (tribromomethane sulfonyl benzene), 10 kg of 20 mass % aqueous solution of modified polyvinyl alcohol (POVAL MP203 manufactured by Kuraray Co., Ltd.), 0.4 kg of 20 mass % aqueous solution of sodium triisopropylnaphthalenesulfonate, and 14 kg water were mixed well to form a slurry. This slurry was sent via a diaphragm pump and dispersed for 5 hours with a horizontal sand mill (trade name: UVM-2, manufactured by AIMEX) charged with zirconia beads having an average diameter of 0.5 mm, and then 0.2 g of benzoisothiazolinone sodium salt and water were added thereto such that the concentration of the organic polyhalogen compound became 26 mass %, to give a dispersion of the organic polyhalogen compound-1. The particles of the organic polyhalogen compound contained in the polyhalogen compound dispersion thus obtained had a median diameter of 0.41 μm and a maximum particle diameter of 2.0 μm or less. The resultant organic polyhalogen compound dispersion was filtered through a polypropylene filter having a pore diameter of 10.0 μm to remove foreign substances such as contaminants, and then stored.

<<Preparation of a Dispersion of an Organic Polyhalogen Compound-2>>

10 kg of an organic polyhalogen compound-2 N-butyl-3-tribromomethanesulfonylbenzamide), 20 kg of 10 mass % aqueous solution of modified polyvinyl alcohol (trade name: POVAL MP203 manufactured by Kuraray Co., Ltd.), and 0.4 kg of 20 mass % aqueous solution of sodium triisopropylnaphthalenesulfonate were mixed well to form a slurry. This slurry was sent via a diaphragm pump and dispersed for 5 hours with a horizontal sand mill (trade name: UVM-2, manufactured by AIMEX) charged with zirconia beads having an average diameter of 0.5 mm, and then 0.2 g of benzoisothiazolinone sodium salt and water were added thereto such that the concentration of the organic polyhalogen compound became 30 mass %. This dispersion was kept warm at 40° C. for 5 hours, to give a dispersion of the organic polyhalogen compound-2. The particles of the organic polyhalogen compound contained in the polyhalogen compound dispersion thus obtained had a median diameter of 0.40 μm and a maximum particle diameter of 1.3 μm or less. The resultant organic polyhalogen compound dispersion was filtered through a polypropylene filter having a pore diameter of 3.0 μm to remove foreign substances such as contaminants, and then stored.

7) Preparation of a Solution of a Phthalazine Compound-1

8 kg modified polyvinyl alcohol MP203 manufactured by Kuraray Co., Ltd. was dissolved in 174.57 kg of water, and then 3.15 kg of 20 mass % sodium triisopropylnaphthalenesulfonate aqueous solution and 14.28 kg of 70 mass % phthalazine compound-1 (6-isopropyl phthalazine) aqueous solution were added thereto to prepare 5 mass % phthalazine compound-1 solution.

8) Preparation of a Mercapto Compound

### <<Preparation of an Aqueous Solution of a Mercapto Compound-1>>

7 g of a mercapto compound-1 (1-(3-sulfophenyl)-5-mercaptotetrazole sodium salt) was dissolved in 993 g of water to give 0.7 mass % aqueous solution.

<<Preparation of an Aqueous Solution of a Mercapto Compound-2>>

20 g of a mercapto compound-2 (1-(3-methylureidophenyl)-5-mercaptotetrazole) was dissolved in 980 g of water to give 2.0 mass % aqueous solution.

9) Preparation of SBR Latex Solution

287 g of distilled water, 7.73 g of surfactant (PIONINE A-43-S, solid content 48.5 mass %, manufactured by Takemoto Oil & Fat Co., Ltd.), 14.06 ml of 1 mol/L NaOH, 0.15 g of tetrasodium ethylenediamine tetraacetate, 255 g of styrene, 11.25 g of acrylic acid, and 3.0 g of tert-dodecylmercaptan were introduced into a polymerization tank in a gas monomer reactor (trade name: TAS-2J, manufactured by Taiatsu Techno Corporation), and the reaction container was hermetically closed, and the mixture was stirred at a stirring rate of 200 rpm. After the reaction container was degassed with a vacuum pump and the internal air was replaced several times by nitrogen gas, 108.75 g of 1,3-butadiene was injected and the internal temperature was elevated to 60° C. A solution of 1.875 g of ammonium persulfate in 50 ml of water was added thereto and stirred for 5 hours. The mixture was further heated to 90° C. and stirred for 3 hours. After the reaction was finished, the internal temperature was lowered to room temperature. Then, the mixture was treated by adding 1 mol/L NaOH and NH₄OH thereto such that Na⁺ ion:NH₄⁺ ion became 1:5.3 (molar ratio) and the pH was adjusted to 8.4. Thereafter, the reaction mixture was filtered through a polypropylene filter having a pore diameter of 1.0 μm to remove foreign substances such as contaminants, to give 774.7 g of SBR latex TP-1.

When the halogen ions were measured by ion chromatography, the concentration of chloride ions was 3 ppm. The concentration of the chelating reagent as determined by high performance liquid chromatography was 145 ppm.

The latex had a gelation degree of 73 mass %, an average particle diameter of 90 nm, Tg=17° C., a solid content of 44 mass %, an equilibrium moisture content of 0.6 mass % at 25° C. under 60% RH, and an ion conductivity of 4.80 mS/cm (ion conductivity was measured at 25° C. with a diagometer CM-30S manufactured by Toa Electronics Ltd.).

### 4-2. Preparation of Coating Solutions

1) Preparation of an Organic Silver Salt-Containing Layer Coating Solution

1000 g of the fatty acid silver salt dispersion obtained above, 135 ml of water, 25 g of the organic polyhalogen compound-1 dispersion, 39 g of the organic polyhalogen compound-2 dispersion, 171 g of the phthalazine compound-1 solution, 1060 g of the SBR latex liquid, 153 g of the reducing agent-2 dispersion, 22 g of the hydrogen bonding compound-1 dispersion, 4.8 g of the reduction accelerator-1 dispersion, 5.2 g of the reduction accelerator-2 dispersion, and 8 ml of the mercapto compound-2 aqueous solution were added successively, then sent to a coating die and applied.

2) Preparation of an Intermediate Layer Coating Solution

27 ml of a 5 mass % aerosol OT (manufactured by American Cyanamid) aqueous solution, 135 ml of a 20 mass % diammonium phthalate aqueous solution and water were added to a mixture of 1000 g of polyvinyl alcohol PVA-205 (manufactured by Kuraray Co., Ltd.), 27 ml of a 5 mass % sodium di(2-ethylhexyl)sulfosuccinate aqueous solution and 4200 ml of a 19 mass % latex liquid of methyl methacrylate/styrene/butyl acrylate/hydroxyethyl methacrylate/acrylic acid copolymer (copolymerization mass ratio: 57/8/28/5/2), such that the total amount became 10,000 g. The resultant mixture was adjusted to pH 7.5 with NaOH, to give an intermediate layer coating solution which was then sent to a coating die to give a coating amount of 8.9 ml/m².

3) Preparation of a Second Protective Layer Coating Solution

100 g of inert gelatin and 10 mg of benzoisothiazolinone were dissolved in 840 ml of water. Then 180 g of a 19 mass % methyl methacrylate/styrene/butyl acrylate/hydroxyethyl methacrylate/acrylic acid copolymer (copolymerization mass ratio: 57/8/28/5/2) latex liquid, 46 ml of a 15 mass % phthalic acid solution in methanol and 5.4 ml of a 5 mass % sodium di(2-ethylhexyl)sulfosuccinate were added to and mixed with the gelatin solution. The resultant mixture just before application was mixed with 40 ml of a 4 mass % chromium alum solution by a static mixer, and was sent to a coating die to give a coating amount of 26.1 ml/m².

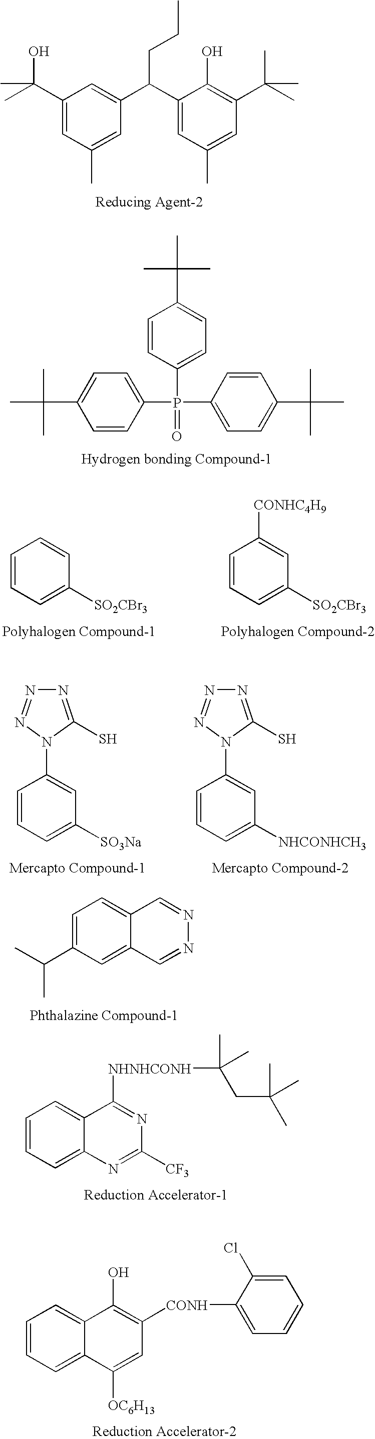

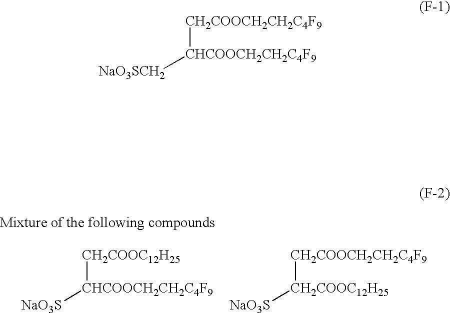

4) Preparation of a First Protective Layer Coating Solution

100 g of inert gelatin and 10 mg of benzoisothiazolinone were dissolved in 800 ml of water and then mixed with 10 g of a 10 mass % emulsion of liquid paraffin, 30 g of a 10 mass % emulsion of dipentaerythritol hexaisostearate, 180 g of a 19 mass % latex liquid for methyl methacrylate/styrene/butyl acrylate/hydroxyethyl methacrylate/acrylic acid copolymer (copolymerization mass ratio: 57/8/28/5/2), 40 ml of a 15 mass % phthalic acid solution in methanol, 5.5 ml of a 1 mass % solution of a fluorine-based surfactant (F-1), 5.5 ml of a 1 mass % aqueous solution of a fluorine-based surfactant (F-2), 28 ml of a 5 mass % aqueous solution of sodium di(2-ethylhexyl)sulfosuccinate, 4 g of polymethyl methacrylate fine particles (average particle diameter 0.7 μm, volume weighted average distribution 30%), and 21 g of polymethyl methacrylate fine particles (average particle diameter 3.6 μm, volume weighted average distribution 60%). The mixture was sent as an outermost layer coating solution to a coating die to give a coating amount of 8.3 ml/m².

### 4-3. Application of an Organic Silver Salt-Containing Sheet

The coating solutions were applied simultaneously by a slide bead coating method onto a temporary support (PET of 100 μm in thickness) to form a multilayer coating consisting of the organic silver salt layer, the intermediate layer, the second protective layer and the first protective layer in this order, whereby a photothermographic material sample was prepared. At this time, the organic silver salt layer coating solution and the intermediate layer coating solution were adjusted to a temperature of 31° C., the second protecting layer coating solution was adjusted to 36° C., and the first protective layer coating solution was adjusted to 37° C.

The coating amounts (g/m²) of the respective compounds in the organic silver salt layer were as follows:

| | |
|---|---|
| Fatty acid silver salt | 3.09 |
| Polyhalogen compound-1 | 0.14 |
| Polyhalogen compound-2 | 0.28 |
| Phthalazine compound-1 | 0.18 |
| SBR latex | 9.43 |
| Reducing agent-2 | 0.77 |
| Hydrogen bonding compound-1 | 0.112 |
| Reduction accelerator-1 | 0.019 |
| Reduction accelerator-2 | 0.016 |
| Mercapto compound-2 | 0.003 |

The coating and drying conditions are as follows:

The coating was carried out at a speed of 160 m/min., the distance between the tip of the coating die and the support was 0.10 mm to 0.30 mm, and the pressure of a decompression chamber was set at a pressure that was lower than atmospheric pressure by 196 Pa to 882 Pa. Before the coating, the support was electrically neutralized with ion wind.

In a subsequent chilling zone, the coating solution was cooled with wind at dry-bulb temperature of 10 to 20° C., then was delivered in a non-contact manner, and was dried with drying air at a dry-bulb temperature of 23 to 45° C. and a wet-bulb temperature of 15 to 21° C. with a coiled non-contact-type drying device.

After the drying, the humidity of the coating was controlled under 40% RH to 60% RH at 25° C., the coating surface was heated to 70 to 90° C. After the heating, the coating surface was cooled to 25° C.

### 4-4. Preparation of a Sensitized Sheet

The resultant coating sample was cut in the size of 200 mm×70 mm, and a polyester pressure-sensitive adhesive tape (No. 31B 71High, manufactured by Nitto Denko Corporation) was attached to the surface of the protective layer. Then the coating layers including the organic silver salt layer, which were attached to the pressure-sensitive adhesive tape, were released from the temporary support, so that a sensitized sheet was obtained.

5. Preparation of Immunochromatography Kits

### 5-1. Construction of Kit A (Comparative Example)

As shown in FIG. 1, the antibody-immobilized membrane 3 was attached to a back pressure-sensitive adhesive sheet 1 (trade name: ARCARE 9020, Nippun TechnoClaster Inc.). At this time, among the two long sides of the membrane, the long side at the anti-hCG antibody line side was arranged downwards. The gold colloid antibody-holding pad 2 was attached onto the antibody-immobilized membrane such that the gold colloid antibody-holding pad 2 overlapped the lower portion of the antibody-immobilized membrane by about 2 mm. Further, a sample addition pad 5 (glass fiber pad cut in the size of 18 mm×150 mm (trade name: GLASS FIBER CONJUGATE PAD, manufactured by Millipore)) was attached onto the gold colloid antibody-holding pad such that the sample addition pad 5 overlapped the lower portion of the gold colloid antibody-holding pad by about 4 mm. An absorbent pad 4 (cellulose membrane cut in the size of 20 mm×150 mm (trade name: CELLULOSE FIBER SAMPLE PAD, manufactured by Millipore)) was attached onto the antibody-immobilized membrane such that the absorbent pad 4 overlapped the upper portion of the antibody-immobilized membrane by about 5 mm. Using a guillotine cutter (trade name: CM4000, manufactured by Nippun TechnoClaster Inc.), the resultant laminated member was cut in parallel to the short side of the laminated member such that the long side of the member was cut at 5 mm intervals, whereby immunochromatographic strips of 5 mm×55 mm were prepared. These strips were placed in a plastic case (Nippun TechnoClaster Inc.) to give an immunochromatography kit A for test. The capture site 3a was composed of a detection part 31 for detecting a sample antibody and a control part 32 for indicating a process noise, and judgment can be made from the difference in coloring (darkening) density between these parts. The region of the antibody-immobilized membrane 3 where the anti-hCG monoclonal antibody for immobilization was applied in a line shape was the detection part 31, and the region wherein the control anti-mouse IgG antibody was applied in a line shape was the control part 32.

### 5-2. Construction of Kit B (According to the Invention)

The antibody-immobilized membrane 3 prepared in item 3 above was attached to a back pressure-sensitive adhesive sheet 1 (trade name: ARCARE 9020, manufactured by Nippun TechnoClaster Inc.). At this time, among the two long sides of the membrane, the long side at the anti-hCG antibody line side was arranged downwards. The sensitized sheet 6 was attached thereon such that the organic silver salt layer surface contacted the surface of the antibody-immobilized membrane. The gold colloid antibody-holding pad 2 prepared in item 2 above was attached onto the antibody-immobilized membrane such that the gold colloid antibody-holding pad 2 overlapped the lower portion of the antibody-immobilized membrane by about 2 mm. A sample addition pad 5 (glass fiber pad cut in the size of 18 mm×150 mm (trade name: GLASS FIBER CONJUGATE PAD, manufactured by Millipore)) was attached onto the gold colloid antibody-holding pad such that the sample addition pad 5 overlapped the lower portion of the gold colloid antibody-holding pad by about 4 mm. An absorbent pad 4 (cellulose membrane cut in the size of 20 mm×150 mm (trade name: CELLULOSE FIBER SAMPLE PAD, manufactured by Millipore)) was attached onto the antibody-immobilized membrane such that the absorbent pad 4 overlapped the upper portion of the antibody-immobilized membrane by about 5 mm. Using a guillotine cutter (trade name: CM4000, manufactured by Nippun TechnoClaster Inc.), the resultant laminated member was cut in parallel to the short side of the laminated member such that the long side of the member was cut at 5 mm intervals, whereby immunochromatographic strips of 5 mm×55 mm were prepared. These strips were placed in a plastic case (Nippun TechnoClaster Inc.) to give an immunochromatography kit B for test.

6. Performance Evaluation

1) Method for Testing Minimum Detectable Sensitivity

hCG (trade name: RECOMBINANT HCG R-506 manufactured by Rohto Pharmaceutical Co., Ltd.) was dissolved in a PBS buffer containing 1 mass % BSA to prepare test hCG solutions at several concentrations.

100 μL of the test hCG solution at each concentration was dropped onto each immunochromatography kit for test. 15 minutes later, the site (capture site) of the antibody-immobilized membrane on which the anti-hCG antibody had been applied was visually checked to judge the degree of coloration according to the following criteria (4 levels):

darkly colored “+++”

colored “++”,

slightly colored “+”; and

uncolored “−”.

The lowest concentration at which the detection was possible was assumed to be the minimum detectable sensitivity for the kit.

In the case of the kit B, 13 minutes after the dropping, the kit B was heated on a hot plate for 30 seconds such that the surface temperature of the kit became 60° C.

2) Results

TABLE 1

| | Degree of Coloration |
|---|---|
| hCG concentration | of Detection Zone |

| (ng/ml) | Kit A | Kit B |
|---|---|---|
| 100.00 | +++ | +++ |
| 30.00 | ++ | +++ |
| 10.00 | + | +++ |
| 3.00 | − | +++ |
| 1.00 | − | +++ |
| 0.30 | − | +++ |
| 0.10 | − | +++ |
| 0.03 | − | + |
| 0.01 | − | − |
| 0.00 | − | − |

As is evident from Table 1, the kit B according to the invention was able to detect hCG with extremely high sensitivity as compared with the comparative kit A.

According to the present invention, the sensitivity of immunochromatography can be improved while maintaining its advantage in easiness and rapidness; in other words, an immunochromatography kit can be provided which enables rapid and easy measurement with higher sensitivity than the measurement with conventionally known immunochromatographic assay kits. According to the present invention, there can be provided a highly sensitive immunochromatography kit which can qualitatively and quantitatively measure an analyte-containing sample easily, rapidly and accurately.

All publications, patent applications, and technical standards mentioned in this specification are herein incorporated by reference to the same extent as if each individual publication, patent application, or technical standard was specifically and individually indicated to be incorporated by reference.

What is claimed is:

1. An immunochromatography kit comprising organic silver salt particles, a reducing agent for silver ions, and a metal colloid label or metal sulfide label,
wherein the immunochromatography kit uses an immune reaction of an analyte and an antibody or antigen that binds specifically to the analyte and analyzes a signal from a label derived from the immobilized immune complex.

2. The immunochromatography kit according to claim 1, further comprising a solvent for the organic silver salt particles.

3. The immunochromatography kit according to claim 1, wherein the organic silver salt particles comprise silver carboxylate or a nitrogen-containing heterocyclic silver salt.

4. The immunochromatography kit according to claim 3, wherein the nitrogen-containing heterocyclic silver salt is a triazole compound or a tetrazole compound.

5. The immunochromatography kit according to claim 3, wherein the nitrogen-containing heterocyclic silver salt is a silver salt of a benzotriazole compound.

6. The immunochromatography kit according to claim 1, wherein the phase transition temperature of the organic silver salt is from 40 to 100° C.

7. The immunochromatography kit according to claim 1, wherein the organic silver salt particles, the reducing agent for silver ions and the metal colloid label or the metal sulfide label are contained in a single component part of the kit.

8. The immunochromatography kit according to claim 7 comprising, in the single component part of the kit, a layer containing the organic silver salt particles and the reducing agent for silver ions and a layer containing the metal colloid label or the metal sulfide label.

9. The immunochromatography kit according to claim 1, wherein the metal colloid is a gold colloid, a silver colloid, a platinum colloid or a composite colloid thereof.

10. The immunochromatography kit according to claim 1, wherein the average particle diameter of the metal colloid is from 5 to 100 nm.

11. The immunochromatography kit according to claim 1, wherein the antibody or antigen is immobilized on a support.

12. The immunochromatography kit according to claim 11, wherein the immune complex comprises the antibody or antigen, the analyte and an additional labeled antibody or antigen.

13. The immunochromatography kit according to claim 11, wherein the immune complex comprises the antibody or antigen and the analyte, wherein the analyte is labeled.
